(19) United States
(12) Patent Application Publication
(10) Pub. No.: US 2024/0398410 A1
(43) Pub. Date: Dec. 5, 2024

(54) END-TO-END CONNECTOR FOR TEXTILE GRAFT ANASTOMOSIS

(71) Applicant: Bipore Medical Devices, Inc., Norwood, NJ (US)

(72) Inventor: Durmus Koch, Englewood, NJ (US)

(73) Assignee: Bipore Medical Devices, Inc., Norwood, NJ (US)

(21) Appl. No.: 18/644,279

(22) Filed: Apr. 24, 2024

Related U.S. Application Data
(60) Provisional application No. 63/469,591, filed on May 30, 2023.

(51) Int. Cl. A61B 17/11; A61B 17/00; A61B 17/04

(52) U.S. Cl. CPC A61B 17/11; A61B 17/0469; A61B 2017/00867; A61B 2017/1132

(57) ABSTRACT

An assembly for an end-to-end anastomosis includes a self-supporting, radially contractible and radially expandable hollow tubular metallic member having opposed open ends. A textile graft is securably disposed over one of the open ends of the hollow tubular member. The other end of the hollow tubular member may be disposed within a bodily lumen for an end-to-end anastomosis. The assembly may further include a suture needle and suture material, where the suture material is securably disposed to a portion of the tubular member.

### CROSS-REFERENCE TO RELATED APPLICATIONS

This application claims the benefit of U.S. Provisional Application No. 63/469,591, filed May 30, 2023, the contents of which is incorporated by reference herein.

### FIELD OF THE INVENTION

The present invention is related to a connector and graft assembly for anastomosis. More particularly, the present invention is related to a connector configured as a coiled tubular member and a textile graft or prosthesis assembly for end-to-end anastomosis.

### BACKGROUND OF THE INVENTION

An anastomosis is typically a surgical connection between two tubular vessels, such as blood vessels. For example, when part a blood vessel is surgically removed, the two remaining ends may be sewn or stapled together, i.e., an end-to-end anastomosis. A side-to-side anastomosis involves a connection between two vessels lying or positioned beside each other. An end-to-side anastomosis involves a connection of the end of one vessel to a side of another vessel. When the vessels are all bodily lumens, any anastomosis typically involves sewing, suturing, or stapling of the vessels.

EP 1411864 B1 describes devices for performing an end-to-end anastomosis requiring multiple tubular guides and stents. Further, the some of the guides or stents require open net-like or grid-like configurations that are disposed over outside of the bodily lumen needing the anastomosis.

U.S. Pat. No. 6,896,687 B2 describes devices for mostly for end-to-side anastomosis, but includes an embodiment for an end-to-end anastomosis. The device for the end-to-end anastomosis is a tubular member having internal barbs for penetrating lumen, in particular, intestinal, walls of the two lumens for anastomosis. A practitioner must force the lumen walls against the internal barbs of the device, thereby making the procedure difficult to preform.

US 2006/0004391 A1 describes a vessel connector which is a hollow tubular device having a plurality of holes or perforations through which a practitioner must loop suture material there through for an anastomosis. Such perforations are often covered by a bodily lumen, thereby making the anastomosis procedure unduly complicated.

As such, there is a need in the art for a simple device of performing and end-to-end anastomosis without having complicated and/or difficult to use structures and/or components.

### SUMMARY OF THE INVENTION

The present invention is directed to an assembly for an end-to-end anastomosis. The assembly provides for an easy-to-use device for use by a practitioner without undue complexity. A suture may be readily secured to the device by a practitioner or may be pre-secured to the device at a tie down portion which is readily available to a practitioner. The suture may have a needle secured thereto.

The assembly for an end-to-end anastomosis according to the present invention includes or comprises a self-supporting, radially contractible and radially expandable hollow tubular member having an outer surface and opposed first and second open ends and an elongate tie down member or raised connector having opposed ends with each of the opposed ends being secured to a portion of the outer surface of the tubular member and a medial portion disposed therein between. The medial portion of the tie down member is raised away from the outer surface of the tubular member. A suture may be secured to the raised medial portion.

The hollow tubular member is desirably in the form of a coil of sheet material rolled into a cylindrical shape. The sheet material may be a solid sheet of material free of perforations and/or free of an open lattice structure. The coil is radially contractible by a compressive force, such as a force applied by a practitioner, where the coil internally and inwardly rolls upon itself to go from an at-rest or quiescent state to a radially compressed state. In such a radially compressed state an artificial graft may be slidingly disposed over an end of the compressed coil.

The hollow tubular member or coil may comprise a metallic material, such as, but not limited to, a shape memory material. The shape memory material may be a nickel-titanium alloy.

The hollow tubular member or coil is self supporting while also having a low profile and/or being thin at a wall or sheet thickness from about 0.001 inches to about 0.003 inches.

The assembly may include a suture having first and second opposed ends and a suture needle. The first end of the suture may be secured to the raised portion of the tie down member and the second end of the suture may be secured to the suture needle. The suture may include a nonabsorbable polymeric material, such as polyamide, polyester, polyvinylidene fluoride, polypropylene, ultra high molecular weight polyethylene, expanded polytetrafluoroethylene, polytetrafluoroethylene, polybutester, and the like. The suture may be monofilament or multifilament suture. The needle may include a metallic needle, such as a stainless steel needle.

The assembly may include a hollow tubular graft having one of its open ends disposed over an open end of the hollow tubular member. When the hollow tubular member is in a radially contractible configuration, the tubular graft is configured to be slidably disposed over the first end of the hollow tubular member. When the hollow tubular member is in a radially expandable or quiescent configuration, the tubular graft is configured to be securably disposed over the end of the hollow tubular member. The graft may be a textile graft, including a crimped textile graft. The graft may further include an inner liner or layer pf polymeric material to provide low permeability to prohibit or avoid leakage of blood or other bodily fluid through the graft wall.

A practitioner may simple move the other end of the hollow tubular member not having the tubular graft into a bodily lumen or move the bodily lumen there over. The practitioner may the suture the bodily lumen to the tubular grant for preform the end-to-end anastomosis.

Thus, the present invention provides a simple and effective device and technique for performing an end-to-end anastomosis.

These and other features and advantages of the present invention will become apparent from the following detailed description of illustrative embodiments thereof, which is to be read in connection with the accompanying drawings. Corresponding reference element numbers or characters indicate corresponding parts throughout the several views of the drawings.

### BRIEF DESCRIPTION OF THE DRAWINGS

FIG. 1 is a perspective view of the hollow tubular member of the end-to-end anastomosis assembly according to the present invention.

FIG. 2 depicts a flat sheet useful for producing the hollow tubular member of FIG. 1.

FIG. 3A is a cross-sectional view to the tubular member of FIG. 1 taken along the 3-3 axis.

FIG. 3B is an exploded view of a portion of the tubular member of FIG. 3A.

FIGS. 4A and 4B are schematic depictions of the tubular member of FIG. 1 having a nominal diameter and a reduced nominal diameter when subjected to a compressive force.

FIG. 5 is a perspective view of a tubular prosthesis used in conjunction with the tubular member of FIG. 1 for providing the end-to-end anastomosis assembly according to the present invention.

FIG. 6 is a cross-sectional view of the tubular prosthesis of FIG. 5 taken along the 6-6 axis.

FIG. 7 is a cross-sectional view of a portion of the tubular prosthesis of FIG. 4 taken along the 7-7 axis.

FIG. 8 is a schematic depiction of the tubular member of FIG. 1 having the tubular prosthesis of FIG. 5 partially disposed there over.

FIG. 9 is a schematic depiction of the end-to-end anastomosis assembly disposed within a bodily lumen.

FIG. 10 is a schematic depiction an end-to-end anastomosis between a bodily lumen and the assembly according to the present invention.

### DETAILED DESCRIPTION OF THE INVENTION

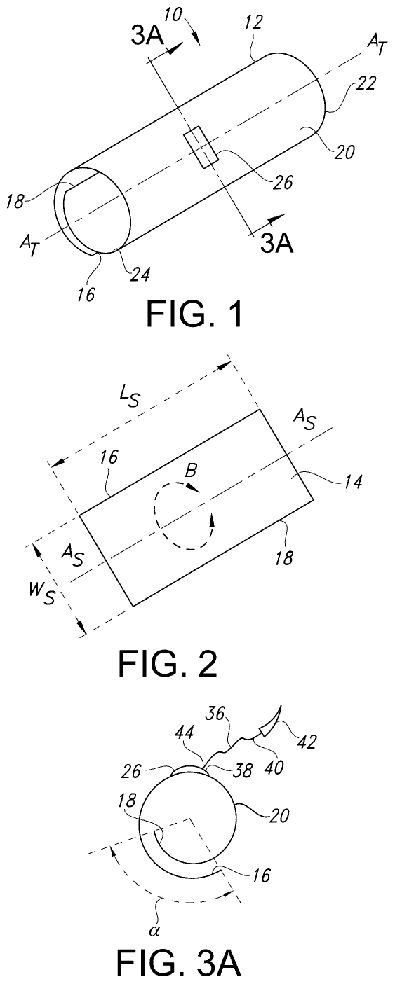

FIG. 1 is a perspective view of a tubular member 12 useful as a connector for an end-to-end anastomosis assembly 10 according to the present invention. The tubular member 12 has a tubular wall 20 and opposed open first and second ends 22, 24. The tubular wall 20 further includes a raised connector or tie down member 26.

The tubular wall 20 may be formed from a sheet material, such as a planar sheet 14 as depicted in FIG. 2. As depicted in FIG. 2, the planar sheet 14 includes a length Ls and a width Ws. Desirably, the dimension or extent of the length Ls is greater that the dimension or extent of the width Ws. The planar sheet 14 may be rolled or coiled about its longitudinal axis As as indicated by arrow B.

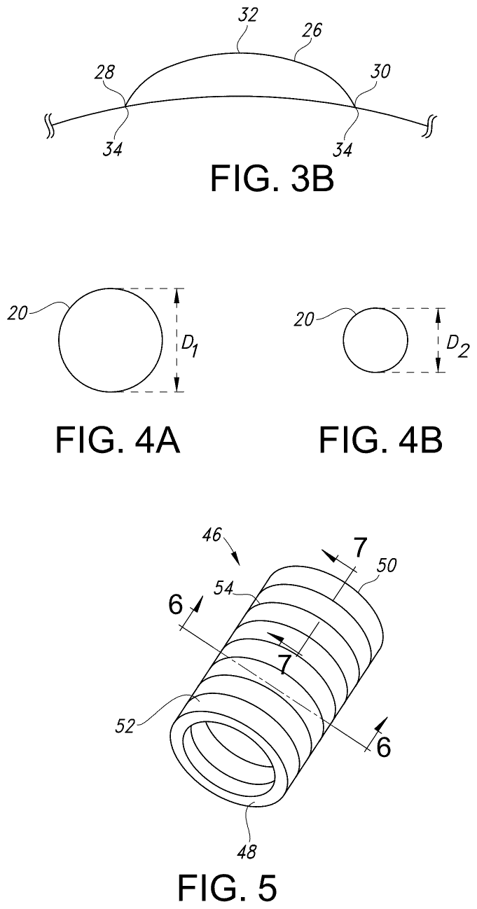

As depicted in FIGS. 1 and 3A, one side edge 18 of the sheet 14 is disposed inwardly to the opposed side edge 16 of sheet 14 when the sheet 14 is rolled or coiled into the tubular wall 20 of the tubular member 12. The tubular member 12 has a longitudinal axis A_{T} as depicted in FIG. 1. The side edges 16 and 18 are slidably disposed about the longitudinal axis A_{T} in an overlapping configuration. An amount or degree of overlap is depicted an angle α in FIG. 3A. The amount or degree of overlap may vary from about 20° to about 340°, more preferably from about 60° to about 240°, even more preferably from about 160° to about 200°, including about 180°.

The side edges 16 and 18 are slidably disposed on the coiled or tubular configuration of the tubular wall 20. As depicted in FIGS. 4A and 4B, the tubular wall 20 may have a nominal diameter D1 in its quiescent state with no compressive force (not shown) being applied and a smaller nominal diameter D2 when a compressive force (not shown) is applied. The compressive force may be applied by an apparatus or individual, such as but not limited to a practitioner, to reduce the nominal diameter for, inter alia, a placement within a textile prosthesis or a bodily lumen.

The tubular member 12 is desirably a planar sheet 14 free or substantially free of openings, holes, or open lattice structures. This aids in prevention of fluid leakage, such as blood leakage, at an end-to-end anastomosis.

The planar sheet material 14 or tubular wall 20 may be made from a thin flexible, metallic material. One useful metallic material is nickel-titanium alloy (NiTi), such as NITINOL. Other materials such as, but not limited to, stainless steel, cobalt-based alloy such as ELGILOY, titanium, tantalum, niobium and combinations thereof. Desirably, the planar sheet 14 or tubular wall 20 is a shape memory material, such as nickel-titanium alloy or NITINOL. Such shape memory material may be heat set into a desired shape, such as the coiled tubular shape described above, by placing the shape memory material into the desired shape and annealing at elevated temperatures, such as 550° C. to 550° C., followed by cooling and quenching. The heat setting conditions are non-limiting and other conditions may suitably be used.

The thickness of the planar sheet 14 of tubular wall 20 may be from about 0.001 inches (or about 0.025 mm) to about 0.003 inches (or about 0.076 mm), more desirable from about 0.002 inches (or about 0.51 mm) to about 0.0025 inches (or about 0.064 mm).

As depicted in FIGS. 1, 3A, and 3B, the tubular member 12 includes an elongate tie down member or a raised connector 26. The raised connector 26 is an elongate member having its opposed first and second ends 28, 30 affixed or secured, for example by welding or brazing, to a portion 34 or portions 34 of the tubular wall 20. The medial portion 32 of the raised connector 26 between it opposed ends 28, 30 is not affixed or secured to the tubular wall 10, but is raised or disposed above the tubular wall 20 as to provide a gap between the medial portion 32 of the raised connector 26 and the tubular wall 20. A suture 36 may have one of its opposed ends such as first end 38, secured to the raised connector 26 by any suitable means, such as tying, knotting, looping, gluing, and the like. The other opposed end, such as second end 40, of the suture 36 may have a suture needle 42 secured thereto or thereat. The suture needle 42 may be of any suitable material, typically stainless

The tubular wall 20 and the raised connector 26 may be made from the same material or may be made from different materials. Desirably, the tubular wall 20 and the raised connector 26 are made from the same material.

Materials for the suture 36 may include natural, synthetic, or polymeric non-absorbable materials. Silk is a non-absorbable suture material derived from an organic protein called fibroin. Synthetic or polymeric materials may include polyamides or nylon (including nylon 6,6), polyesters or polyethylene terephthalate (PET), polyvinylidene fluoride (PVDF), polypropylene, ultra high molecular weight polyethylene (UHMWPE), expanded polytetrafluoroethylene (ePTFE), polytetrafluoroethylene (PTFE), and polybutester (copolymer of polyglycol terephthalate and polybutylene terephthalate). The sutures may be monofilament or multifilament, including twisted or braided multifilament. Desirably, suture 36 is made of non-absorbable materials.

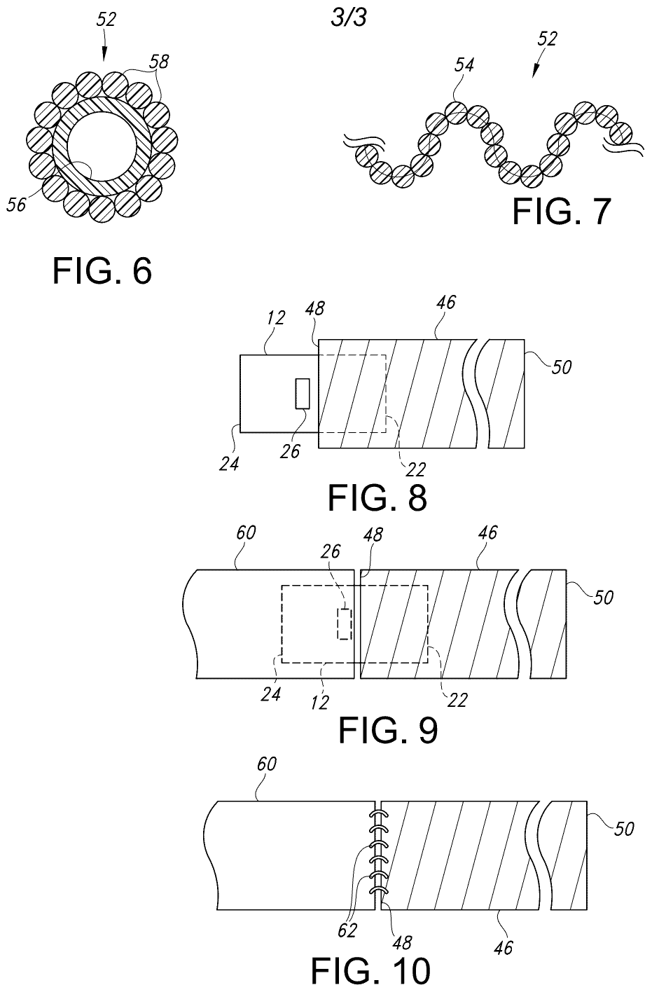

The tubular member 12 is useful for providing an end-to-end anastomosis between a bodily lumen and an artificial tubular member. A useful artificial tubular member is depicted as a tubular textile prosthesis 46 in FIG. 5. The prosthesis 46 has opposed open ends 48, 50 and textile wall 52. The textile wall 52 may include crimps 54 as depicted in FIG. 7, which is a partial cross-sectional view of the textile wall 52 taken along the 7-7 axis in FIG. 5. A cross-sectional view of the prosthesis 46 taken along the 6-6 axis in FIG. 5 is depicted in FIG. 6. The textile wall 52 includes a plurality of yarns 58. An inner liner or layer 56 may optionally be securably disposed within the prosthesis 46.

The textile wall 52 of the prosthesis 46 may include wall portions made from any biocompatible, durable material, including, for example polyesters, such as polyethylene terephthalate (PET); naphthalene dicarboxylate derivatives, such as polyethylene naphthalate, polybutylene naphthalate, polytrimethylene naphthalate, trimethylenediol naphthalate; polytetrafluoroethylenes (PTFE); expanded polytetrafluoroethylene (ePTFE); polyurethanes; polyamides; polyimides; polycarbonates; natural silk; polyethylene; polypropylene; and combinations thereof. As used herein, textile materials are filaments or yarns that are woven, braided, knitted, filament-spun, and the like to form textile graft material. Desirably, the textile wall 52 of the prosthesis 46 is a woven poly(ethylene terephthalate) or a woven PET textile tubular member.

The yarns 58 may be of the monofilament, multifilament, or spun type. The yarns may have a linear density from about 18 denier (about 20 decitex) to about 140 denier (about 154 decitex). The yarns may be flat, twisted, and/or textured, and may have high, low or moderate shrinkage and/or bulk and crimp properties. Twisted yarns include S-twisted yarns and Z-twisted yarns.

The textile wall 52 of the prosthesis 46 may be woven from yarns using any known weave pattern, including simple plain weaves, basket weaves, twill weaves, velour weaves and the like. Weave patterns include warp yarns running along the longitudinal length of the woven product and weft also known as fill yarns running around the width or circumference of the woven product. The warp and the fill yarns are at approximately 90 degrees to one another with fabric flowing from the machine in the warp direction.

Knitting involves the interlooping or stitching of yarn into vertical columns (wales) and horizontal rows (courses) of loops to form the knitted fabric structure. In warp knitting, the loops are formed along the textile length, i.e., in the wale or warp direction of the textile. Useful knitting patterns include, but are not limited to, locknit knits (also referred to as tricot or jersey knits), reverse locknit knits, sharkskin knits, queenscord knits, atlas knits, velour knits, and the like.

The inner liner or layer 56 may include polytetrafluoroethylene (PTFE) or expanded polytetrafluoroethylene (ePTFE). The inner liner or layer 56 may have a non-limiting thickness from about 50 micrometers (0.002 inches or 2 mil) to about 75 micrometers (0.003 inches or 3 mil).

Expanded PTFE (ePTFE) tubes and films may be made by extrusion of PTFE resin, typically mixed with a lubricant. The microporous structure of ePTFE may be obtained by a process that involves rapid stretching of the extruded tube at high temperature. The ePTFE structure may be characterized by nodes, about 5-10 μm wide by about 5-100 μm long, interconnected by fibrils of less than about 0.5 μm in diameter. The degree of porosity of an ePTFE graft is controlled by the distance between the nodes. In clinically used grafts this distance may be in of the order of about 30 μm.

The inner liner or layer 56 may include microporous PTFE substantially free of the node and fibril structure associated with typical ePTFE. Such microporous PTFE may be made by calendering and stretching PTFE films. For vascular applications the average pore size may be less than about 20 μm, including from about 1 μm to about 5 μm.

With the use of the inner liner or layer 56, the prosthesis 46 may be a fluid tight implantable prosthesis which configured to obviate the leaking of blood at a blood pressure of up to approximately 300 mmHg. There is no need for preclotting with collagen or the like as the inner liner or layer 56 is generally blood impermeable. Such a fluid tight implantable prosthesis may have a water permeability of about 0.16 ml/min/cm² at 120 mm Hg pressure or less than 0.16 ml/min/cm² at 120 mm Hg pressure. The use of the inner liner or layer 56 also offers other benefits. For example, PTFE and ePTFE layers also prevent or inhibit or minimize undesirable adhesion or build-up of materials, such as thrombus formation, platelet aggregation, and the like. The inner liner of layer 56 may be secured to the prosthesis 46 by thermoplastic or adhesive means or materials, such as use of thermoplastic elastomers such as polyether block amide (PEBAX) or polyethylene (PE), or use of adhesives such as polycarbonate-urethane or fluorinated ethylene propylene (FEP). These materials are non-limiting, and other suitable biocompatible materials may be used.

FIG. 7 is a cross-sectional view of a portion of the textile wall 22 of FIG. 5 taken along the 7-7 axis. The textile wall 52 includes a plurality of crimps 54 characterized by raised portions (peaks) and lowered portions (valleys). The tubular wall 52 may be crimped prior to securement to the underlying liner or layer 56 of FIG. 6.

As depicted in FIG. 8, one end 22 of the tubular member 12 may be disposed within one end 48 of the prosthesis 46 to form the end-to-end anastomosis assembly 10 of the present invention. The tubular member 12 is securably disposed within the one end 48 of the prosthesis 46 by, for example, compressing the tubular member 12 to a diameter less than the inner diameter of the prosthesis 46 and the removing the compressive force to allow the tubular member 12 to expand against the prosthesis 46, thereby being securably disposed therein. While the prosthesis 46 is depicted as having only one tubular member 12, the present invention is not so limited. For example, either end 48, 50 or both ends 48, 50 may have a tubular member 12 or tubular members 12.

As depicted in FIG. 9, a portion of the end-to-end anastomosis assembly 10 may be disposed within a bodily lumen 60. In particular, the end 24 of the tubular member 12 not having the tubular prosthesis 46 disposed there over is placed with the bodily lumen 60. As depicted in FIG. 10, after such placement, sutures 62 may be sued to secure the bodily lumen 60 to the prosthesis 46. The end-to-end anastomosis assembly 10 may be used or deployed through surgical techniques or percutaneous endovascular techniques. Sutures 62 may be the same as suture 36 described above or may be different or in place thereof.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

Element reference numbers, letters, and/or symbols in the following embodiments or aspects of the present invention are presented merely for ease of comprehension and are not to be construed as limiting the scope of the present invention. Further, the following embodiments or aspects of the invention may be combined in any fashion and combination and be within the scope of the present invention, as follows:

Embodiment 1. An assembly (10) for an end-to-end anastomosis comprising:
a self-supporting, radially contractible and radially expandable hollow tubular member (12) having an outer surface and opposed first and second open ends (22, 24);
an elongate tie down member (26) having opposed ends (28, 30) with each of the opposed ends (28, 30) being secured to a portion (34) of the outer surface of the tubular member (12) and a medial portion (32) disposed thereinbetween,
wherein the medial portion (32) of the tie down member (26) is raised away from the outer surface of the tubular member (12).

Embodiment 2. The assembly (10) of embodiment 1, wherein the hollow tubular member (12) comprises a coil of sheet material (14, 20) rolled into a cylindrical shape.

Embodiment 3. The assembly (10) of embodiment 1 or any previous embodiments, wherein the sheet material (14, 20) is a solid sheet of material free of perforations.

Embodiment 4. The assembly (10) of embodiment 1 or any previous embodiments, wherein the sheet material (14, 20) is a solid sheet of material free of an open lattice structure.

Embodiment 5. The assembly (10) of embodiment 2 or any previous embodiments, wherein the hollow tubular member (12) comprises a metallic material.

Embodiment 6. The assembly (10) of embodiment 5, wherein the metallic material comprises a shape memory material.

Embodiment 7. The assembly (10) of embodiment 6, wherein the shape memory material comprises nickel-titanium alloy.

Embodiment 8. The assembly (10) of embodiment 5 or any previous embodiments 6-7, wherein the metallic material has a thickness from about 0.001 inches to about 0.003 inches.

Embodiment 9. The assembly (10) of embodiment 1 or any previous embodiments, further comprising a suture (36) having first and second opposed ends (38, 40) and a suture needle (42), wherein the first end of the suture (38) is secured to the tie down member (26) and the second end (40) of the suture (36) is secured to the suture needle (42).

Embodiment 10. The assembly (10) of embodiment 9, wherein the suture (36) comprises a nonabsorbable polymeric material.

Embodiment 11. The assembly (10) of embodiment 9 or 10, wherein the suture (36) comprises a polymeric material selected from the group consisting of polyamides, polyesters, polyvinylidene fluoride, polypropylene, ultra high molecular weight polyethylene, expanded polytetrafluoroethylene, polytetrafluoroethylene, and polybutester.

Embodiment 12. The assembly (10) of embodiment 9 or any previous embodiments 10-11, wherein the suture (36) comprises a monofilament or multifilament suture.

Embodiment 13. The assembly (10) of embodiment 9 or any previous embodiments 10-12, wherein the needle (42) comprises stainless steel.

Embodiment 14. The assembly (10) of embodiment 1 or any previous embodiments, further comprising a tubular graft (46) having opposed first and second open ends (48, 50); wherein the first open end (48) of the tubular graft (46) is disposed over the first end (22) of the hollow tubular member (12).

Embodiment 15. The assembly (10) of embodiment 14 or any previous embodiments, wherein, when the hollow tubular member (46) is in a radially contractible configuration, the tubular graft (46) is configured to be slidably disposed over the first end (22) of the hollow tubular member (12).

Embodiment 16. The assembly (10) of embodiment 14 or any previous embodiments, wherein, when the hollow tubular member (12) is in a radially expandable configuration, the tubular graft (46) is configured to be securably disposed over the first end (22) of the hollow tubular member (120).

What is claimed is:

1. An assembly for an end-to-end anastomosis comprising:
a self-supporting, radially contractible and radially expandable hollow tubular member having an outer surface and opposed first and second open ends;
an elongate tie down member having opposed ends with each of the opposed ends being secured to a portion of the outer surface of the tubular member and a medial portion disposed thereinbetween,
wherein the medial portion of the tie down member is raised away from the outer surface of the tubular member.

2. The assembly of claim 1, wherein the hollow tubular member comprises a coil of sheet material rolled into a cylindrical shape.

3. The assembly of claim 1, wherein the sheet material is a solid sheet of material free of perforations.

4. The assembly of claim 1, wherein the sheet material is a solid sheet of material free of an open lattice structure.

5. The assembly of claim 2, wherein the hollow tubular member comprises a metallic material.

6. The assembly of claim 5, wherein the metallic material comprises a shape memory material.

7. The assembly of claim 6, wherein the shape memory material comprises nickel-titanium alloy.

8. The assembly of claim 5, wherein the metallic material has a thickness from about 0.001 inches to about 0.003 inches.

9. The assembly of claim 1, further comprising a suture having first and second opposed ends and a suture needle, wherein the first end of the suture is secured to the tie down member and the second end of the suture is secured to the suture needle.

10. The assembly of claim 9, wherein the suture comprises a nonabsorbable polymeric material.

11. The assembly of claim 9, wherein the suture comprises a polymeric material selected from the group consisting of polyamides, polyesters, polyvinylidene fluoride, polypropylene, ultra high molecular weight polyethylene, expanded polytetrafluoroethylene, polytetrafluoroethylene, and polybutester.

12. The assembly of claim 9, wherein the suture comprises a monofilament or multifilament suture.

13. The assembly of claim 9, wherein the needle comprises stainless steel.

14. The assembly of claim 1, further comprising a tubular graft having opposed first and second open ends; wherein the first open end of the tubular graft is disposed over the first end of the hollow tubular member.

15. The assembly of claim 14, wherein, when the hollow tubular member is in a radially contractible configuration, the tubular graft is configured to be slidably disposed over the first end of the hollow tubular member.

16. The assembly of claim 14, wherein, when the hollow tubular member is in a radially expandable configuration, the tubular graft is configured to be securably disposed over the first end of the hollow tubular member.
